(19) United States
(12) Patent Application Publication
(10) Pub. No.: US 2021/0353517 A1
(43) Pub. Date: Nov. 18, 2021

(54) PROCESS FOR PRODUCING A BIO-BASED SURFACTANT

(71) Applicants: University College Dublin, Dublin 4 (IE); The Provost, Fellows, Scholars and Other Members of Board of Trinity College Dublin, Dublin, 2 (IE)

(72) Inventors: Ramesh BABU, Dublin 15 (IE); Federico CERRONE, Blackrock, Co. Dublin (IE); Shane KENNY, Co. Dublin (IE); Kevin O'CONNOR, Co. Dublin (IE); Jasmina RUNIC, Belgrade (RS)

(21) Appl. No.: 17/278,301

(22) PCT Filed: Sep. 19, 2019
(86) PCT No.: PCT/EP2019/075240

§ 371 Date: Mar. 20, 2021

(30) Foreign Application Priority Data
Sep. 21, 2018 (GB) 1815390.8

(51) Int. Cl. A61K 8/46; C11D 1/16; A61Q 5/02; A61Q 19/10; C07C 303/24; A61Q 11/00; C12P 7/64

(52) U.S. Cl. CPC A61K 8/463; C11D 1/16; A61Q 5/02; C12P 7/6409; C07C 303/24; A61Q 11/00; A61Q 19/10

(57) ABSTRACT

A process for producing a bio-based surfactant comprising an alkyl disulphate salt comprises the steps of methanolysis of medium chain length polyhydroxyalkanoic acid (mcl-PHA) to provide hydroxy fatty acid methyl ester monomers (HFAME's), reduction of the HFAME's to provide 1,3 alkyl diols, sulphation of the 1,3 alkyl diols to provide 1,3 alkyl disulphates, and neutralisation of the alkyl disulphates to provide a bio-based surfactant comprising 1,3 alkyl disulphate salt. A bio-based surfactant comprising a mixture of medium chain length 1,3 alkyl disulphate salts is also described.

### FIELD OF THE INVENTION

The present invention relates to a process for producing a bio-based surfactant. Also contemplated are bio-based surfactants made using the process of the invention, and intermediates generated in the process.

### BACKGROUND TO THE INVENTION

Currently worldwide production of surfactants utilises coconut and palm oil derived fatty acids methyl esters (FAMEs) as raw materials. Both are non-European/US resources and Governments in both regions are driving the use of local renewable resources to produce bio-based products (bioeconomy). Palm oil has a very bad environmental record promoting deforestation and Green house Gas production.

Bio-based surfactants (Sophorolipids, Rhamnolipids) of microbial origin are just appearing recently on the market—the former already commercially available, while the latter still at an early stage development. Sophorolipids are commercialised by Ecover™, Saraya™, Intobio™, Evonik™ and Allied Carbon Solutions™. All of them have CMC 7-10 fold less than CMC of SDS, but need to be produced with yeasts (average fermentations times: 7 days) and with fatty acids of tropical plant origin, that still pose an environmental pressure, due to deforestation issues. In addition, the separation of sophorolipids from the fermentation broth is challenging, and the accumulation of sophorolipids in media creates difficulties with producing the strain, due to lack of oxygen transfer. If they are continuously separated by increasing or decreasing the temperature (to crystallise them) this increases the costs of the process. Rhamnolipids on the contrary are produced effectively by bacterial fermentation (3 days average fermentation time) but only by pathogenic bacteria and are difficult to scale-up (the full bio-based surfactant production process is driven enzymatically with the intervention of 5 energetically expensive enzymes). The latter can therefore only be produced by heterologous production with genetic modification of a non-pathogenic strain. Furthermore, as these rhamnolipids are of pathogenic origin, there is a risk that these compounds could induce an inflammatory response on the mammalian tissues. The CMC for rhamnolipids is 10-11 fold lower than SDS. Other two examples of biopolymeric surfactants are represented by the ones commercially available through WheatOleo™/Soliance™ and by Synthezime™. The formers produce bio-based surfactant made of an alkylpolypentoside synthesized by a chemical condensation of an aliphatic chain with hemicellulosic material that requires quite high temperatures to be achieved (90-150 C). The second company utilise highly genetically modified yeasts to produce a terminally hydroxylated tetradecanoic acid and convert it chemically into a tri/dicarboxylic acid with surfactant properties. These compounds are modified with fossil-based compounds.

Rhamnolipids are produced only by pathogenic bacteria and are difficult to scale-up (the full bio-based surfactant production process is driven enzymatically with the intervention of 5 energetically expensive enzymes), if heterologous production is considered instead, that reduces the productivity and increase considerably the costs. Alkylpolypentosides require high temperature for the chemical synthesis (90-150° C.). Partially biobased tri/dicarboxylic acids are chemically synthesized with the use of fossil derived compound.

It is an object of the invention to provide a bio-based surfactant that overcomes at least one of the above-referenced problems, and in particular to provide a bio-based surfactant that is not reliant on palm or coconut oils, but can be produced from plant oil indigenous to Europe and the US such as sunflower and other plant oils.

### SUMMARY OF THE INVENTION

The present invention addresses the need for a bio-based surfactant that can be generated from hydrolysed plant oils, oils that can be grown in Europe and the US such as sunflower and rapeseed oil, and that is not reliant on using palm and coconut oil which is not indigenous to Europe and US. The bio-based surfactant is produced in a chemo-biotechnological process that employs medium chain length polyhydroxyyalkanoic acid (mcl-PHA), a biopolyester that is produced by microbial fermentation using fatty acids or sugars as a substrate. The process involves the steps of methanolysis (depolymerisation) of the mcl-PHA to produce hydroxy fatty acid methyl esters (HFAME's), which are reduced and sulphated to provide a mixture of alkyl disulphates, and then neutralised to provide the alkyl disulphate salt surfactant. The bio-based surfactant produced has been shown to have bio-based surfactant properties (wettability, surface tension decrease and foaming stability) that are five-fold better than the commercially available sodium dodecyl sulphate when tested at the same concentration.

The invention broadly provides a process for making an alkyl disulphate salt bio-based surfactant, an alkyl disulphate salt bio-based surfactant, compositions comprising the bio-based surfactant, and intermediates produced in the process of the invention (for example a mixture of medium chain length alkyl (1,3) diols.

Process

According to a first aspect of the present invention, there is provided a process for producing a bio-based surfactant comprising an alkyl disulphate salt, comprising the steps of:
methanolysis of medium chain length polyhydroxyalkanoic acid (mcl-PHA) to provide hydroxy fatty acid methyl ester monomers (HFAME's);
reduction of the HFAME's to provide 1,3 alkyl diols;
double sulfation of the 1,3 alkyl diols to provide 1,3 alkyl disulphates; and
neutralisation of the alkyl disulphates to provide a bio-based surfactant comprising 1,3 alkyl disulphate salt.

In one embodiment, the process comprises an initial step of microbial fermentation of fatty acids to produce the mcl-PHA. Typically, the fatty acids are of hydrolysed plant oil origin.

In one embodiment, the mcl-PHA comprises C12 hydroxyalkanoic acid of at least 15 mol % of the polymer, for example 15-50, 15-40, 15-30, or 15-25 mol %.

In one embodiment, the mcl-PHA comprises, or consist essentially of, C6, C8, C10 and C12 hydroxyalkanoic acids in the polymer. Typically, at least 80, 85, 90 or 95 mol % of the polymer is C6, C8, C10 and C12 hydroxyalkanoic acids.

In one embodiment, the mcl-PHA polymer comprises a mol % ratio of C6, C8, C10 and C12 hydroxyalkanoic acids of about 1-90:1-90:1-90:15-97, typically 1-10:10-60:10-60:15-79, typically about 1-5:30-50:30-50:15-40. In one embodiment, the mcl-PHA polymer comprises a mol % ratio of C6, C8, C10 and C12 hydroxyalkanoic acids of about 2:38:40:20.

In one embodiment, the methanolysis step employs methanol and sulphuric acid, typically in a volumetric ratio of about 60-95:5-40, more preferably about 80-90:10-20, and more preferably about 85:15 methanol to sulphuric acid.

In one embodiment, the reduction step employs a borohydride salt, typically sodium borohydride. In one embodiment, the molar ratio of borohydride salt to HFAME is 3:2 to 5:2, preferably about 4:2. Typically, the reduction step is performed in tert butanol with an excess of methanol as the hydrogen donor molecules.

In one embodiment, the double sulfation step comprises reacting the mixture of alkyl diols with chlorosulfonic acid in a suitable volatile solvent (such as diethyl ether).

In one embodiment, the neutralisation employs a suitable base, for example an alkali metal hydroxide, for example sodium hydroxide. When the latter is employed, the alkyl disulphate salt is sodium alkyl disulphate. Other bases, or indeed alkali metal hydroxides, may be employed. Generally the base is employed in equimolar amounts to the alkyl disulphates.

Surfactant

The invention also provides a surfactant comprising a mixture of medium chain length alkyl disulphate salts, typically 1,3 alkyl disulphate salts.

In one embodiment, the surfactant comprises, or consists essentially of, C6, C8, C10 and C12 alkyl disulphate salts.

In one embodiment, the surfactant comprises, or consists essentially of, C6, C8, C10 and C12 1,3 alkyl disulphate salts.

In one embodiment, the C12 alkyl disulphate salt comprises at least 15 mol % of the mixture.

In one embodiment, a mol % ratio of C6, C8, C10 and C12 alkyl disulphate salts in the mixture is about 1-10:10-60:10-60:15-79, typically about 1-5:30-50:30-50:15-40.

In one embodiment, a mol % ratio of C6, C8, C10 and C12 alkyl disulphate salts in the mixture is about 2:38:40:20.

Compositions

The invention also provides a composition comprising a bio-based surfactant of the invention

In one embodiment, the composition is a detergent composition.

In one embodiment, the composition additionally comprises one or more of a non-ionic surfactant, additional anionic surfactant, and a co-surfactant. In one embodiment, the co-surfactant is selected from an amphoteric surfactant, a zwitterionic surfactant, a cationic surfactant, or a mixture thereof.

In one embodiment, the composition is selected from a personal care product, a fabric washing product, a dishwashing product, and a household care product. Exemplary compositions include a liquid, solid or semi-solid soap, fabric washing product, dishwashing product, shampoo, shower or body gel, household cleaning detergent, and toothpaste.

Intermediates

The invention also provides an intermediate formed in the process of the invention, comprising or consisting essentially of a mixture of medium chain length alkyl (1,3) diols.

The process typically comprises the steps of methanolysis of medium chain length polyhydroxyalkanoic acid (mcl-PHA) to provide hydroxy fatty acid methyl ester monomers (HFAME's), and reduction of the HFAME's to provide 1,3 alkyl diols.

In one embodiment, the intermediate comprises C6, C8, C10 and C12 alkyl (1,3) diols.

In one embodiment, the intermediate consists essentially of C6, C8, C10 and C12 alkyl (1,3) diols.

In one embodiment, the mixture comprises at least 15 mol % of C12 alkyl (1,3) diols.

In one embodiment, a mol % ratio of C6, C8, C10 and C12 alkyl (1,3) diols in the mixture is about 1-10:10-60:10-60:15-79, typically about 1-5:30-50:30-50:15-40.

In one embodiment, a mol % ratio of C6, C8, C10 and C12 alkyl disulphate salts in the mixture is about 2:38:40:20.

Other aspects and preferred embodiments of the invention are defined and described in the other claims set out below.

### BRIEF DESCRIPTION OF THE FIGURES

FIG. 1 Chemical synthesis route from hydroxydodecyl acid methyl ester (a) to 1,3 dodecandiol (b) to dodecyl-1,3-disulfate (c).

FIG. 2 Chemical synthesis route from HFAME moiety (a) to alkyldiol moiety (b) to alkyl-1,3-disulfate moiety (c) with each molecule respective percentage ratio (%) in the moiety. The reduction of the HFAME moiety and the sulfation of the alkyldiols products used the same reagents as in FIG. 1

FIG. 3 1H-NMR spectra of 1,3 dodecanediol.

FIG. 4 1H-NMR spectra of sodium dodecyldisulfate.

FIG. 5 1H-COSY NMR of sodium dodecyldisulfate.

FIG. 6 heteronuclear 1H-13C two dimensional HSQC for sodium dodecyldisulfate.

FIG. 7 FTIR spectra of sodium dodecyldisulfate.

FIG. 8 FTIR spectra of HFAME (C12-rich) reduced to an alkyldiols moiety.

FIG. 9 FTIR spectra of alkyldisulphate moiety.

FIG. 10 1H-COSY NMR of sodium alkyldisulfate moiety. The peak at 4.87 is the signal coming from the water chemical shift when in deuterated methanol. (Fulmer et al 2010).

FIG. 11 13C NMR spectra of sodium alkyldisulfate moiety. The biggest peaks are attributed to the H in the deuterated methanol solvent.

FIG. 12. Surface tension curves of the three anionic surfactants (the insert is a magnification of the x-axis area between 0 and 0.25%)

FIG. 13 Conductivity of different surfactant solutions (SDS, dodecyl 1,3-disulfates and alkyldisulfates) at increasing concentration (g/L). The point where the slope of the linear curves intercepting the regression plots change is the CMC for the specific surfactant solution. Specific linear interpolating curves are plotted against the scatter plots.

FIG. 14. Dynamic variation of the contact angle of a water droplet due to the addition of a drop of a specific anionic surfactant (at its CMC) over time.

FIG. 15 Foam stability of the three different anionic surfactants (sodium dodecyldisulfate, sodium alkyldisulfate and SDS) over time.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “bio-based surfactant” is to be read to refer to a surfactant that is produced from mcl-PHA, in which the mcl-PHA is accumulated by bacteria in a microbial fermentation process typically using hydrolysed fatty acid or sugar as a substrate. In the embodiments described herein, the bio-based surfactant comprises a mixture of C6 to C12 1,3 alkyl disulphate salts, chemically derived from mcl-PHA accumulated by a strain of Pseudomonas chlororaphis 555 using rapeseed oil fatty acids as a substrate.

As used herein, the term “alkyl disulphate salt” is to be read to refer to a salt, typically a sodium salt, of an alkyl disulphate typically produced by double sulfation of an alkyl diol and subsequent neutralisation. Typically, the alkyl disulphate is a 1,3 alky disulphates. In one embodiment, the alkyl group is a C6, C8, C10 or C12 hydrocarbon chain, which may be saturated and partially unsaturated. In one embodiment, the salt is a sodium salt, although other alkali metals may be employed, for example potassium.

As used herein, the term “methanolysis” is to be read to refer to a process including coincident steps of hydrolysis and methylation, in which the PHA is depolymerised producing hydroxy fatty acid methyl ester monomers. The methanolysis step typically has a yield of greater than 90% and preferably greater than 95%. In one embodiment, the methanolysis step employs methanol and an acid, typically a strong acid, preferably sulphuric acid although other strong acids such as hydrochloric acid and perchloric acid may be employed. The methanol and acid are employed at a volumetric ratio of about 60-95:5-40, more preferably about 80-90:10-20, and more preferably about 85:15 methanol to acid.

As used herein, the term “medium chain length polyhydroxyalkanoic acid” or “mcl-PHA” refers to linear polyesters having an average monomer chain length of C6 to C14, or C6 to C12. These biopolyesters are accumulated during bacterial fermentation of a suitable substrate, typically sugars or lipids. In one embodiment, the mcl-PHA is substantially non-crystalline, and typically has a crystallinity of less than 30% as determined by a method of x-ray diffraction. Methods of producing mcl-PHA are described in the literature, including Walsh et al (2015), Lee et al (2000), and Madison et al (Microbiology and Molecular Biology Reviews, March 1999, P21-53) in which mcl-PHA is referred to as msc-PHA and formation by Pseudomonas from fatty acids is described on pages 39 and 40. In one embodiment, the mcl-PHA comprises a mixture of C6, C8, C10 and C12 hydroxyalkanoic acids. In one embodiment, the mixture comprises 1-90 mol % C6. In one embodiment, the mixture comprises 1-90 mol % C8. In one embodiment, the mixture comprises 1-90 mol % C10. In one embodiment, the mixture comprises 15-97 mol % C12. In one embodiment, the mcl-PHA polymer comprises a mol % ratio of C6, C8, C10 and C12 hydroxyalkanoic acids of about 1-10:10-60:10-60:15-79, typically about 1-5:30-50:30-50:15-40. In one embodiment, the mcl-PHA polymer comprises a mol % ratio of C6, C8, C10 and C12 hydroxyalkanoic acids of about 2:38:40:20. The alkyl chains may be full saturated and partially unsaturated. The occurrence and degree of unsaturation will depend on the type of substrate employed to produce the mcl-PHA.

As used herein, the term “hydroxy fatty acid methyl ester” or “HFAME” is to be read to refer to the monomer product of the methanolysis of mcl-PHA. Typically, the HFAME is a (R)-3-HFAME, having an average monomer chain length of C6 to C14 or C6 to C12. In one embodiment, the HFAME is a mixture of C6 to C12 HFAME's.

As used herein, the term “reduction” is to be read to refer to the process in which the polar carboxylate group of the HFAME is reduced using a suitable reductant such as sodium borohydride to produced an alkyl (1,3) diol, or a when a mixture of HFAME's is reduced, a mixture of alkyl (1,3) diols.

As used herein, the term “1,3 alkyl diol” is to be read to refer to the product of the reduction of the (R)-3-HFAME. Typically, the 1-3 alkyl diol has an average monomer chain length of C6 to C14 or C6 to C12. In one embodiment, the 1-3 alkyl diol is a mixture of C6 to C12 1-3 alkyl diols.

As used herein, the term “mol %” is to be read to refer to the percentage contribution of each monomer to the composition of the molecular mass of the polymer.

As used herein, the term “microbial fermentation” is to be read to refer to the process by which mcl-PHA is produced. Examples of the use of microbial fermentation to produce mcl-PHA are known from the literature (Walsh et al, Lee et al, etc). In one embodiment, microbial fermentation employs a Pseudomonas strain of bacteria, for example a Pseudomonas putida sub-species such as KT2440, CA-3, G016, and Pseudomonas chlororaphis 555.

As used herein, the term “C12” as applied to an alkyl chin means an alkyl side chain of 12 carbons. The terms “C6”, “C8”, and “C10” should be construed accordingly.

As used herein, the term “hydrolysed plant origin” is to be read to refer to a substrate for use in the production of mcl-PHA by microbial fermentation. Examples include plant oils, especially high oleic acid plant oils such as sunflower and rapeseed oil. Prior to microbial fermentation the plant oils are hydrolysed to release fatty acids from the oil. Fatty acids produced by hydrolysis of plant oils are commercially available.

As used herein, the term “detergent composition” is to be read to refer to a composition comprising a surfactant, for example an anionic surfactant, non-ionic surfactant, or a co-surfactant such as a cationic surfactant, zwitterionic surfactant, or an amphoteric surfactant. The detergent composition may be a household care product, a personal care product, a fabrics cleaning product, or a dishwash product. The detergent composition may also include one of more of a builder, a bleaching agent, a protease enzyme, a perfume, and a fluorescent agent optical brightener). Suitable anionic detergent compounds which may be used are usually water-soluble alkali metal salts of organic sulphates and sulphonates having alkyl radicals containing from about 8 to about 22 carbon atoms, the term alkyl being used to include the alkyl portion of higher alkyl radicals. Examples of suitable synthetic anionic detergent compounds are sodium and potassium alkyl sulphates, especially those obtained by sulphating higher Cs to Cie alcohols, produced for example from tallow or coconut oil, sodium and potassium alkyl C9 to C20 benzene sulphonates, particularly sodium linear secondary alkyl C10 to Ci5 benzene sulphonates; and sodium alkyl glyceryl ether sulphates, especially those ethers of the higher alcohols derived from tallow or coconut oil and synthetic alcohols derived from petroleum. The anionic surfactant is preferably selected from: linear alkyl benzene sulphonate; alkyl sulphates; alkyl ether sulphates; soaps; alkyl (preferably methyl) ester sulphonates, and mixtures thereof. The most preferred anionic surfactants are selected from: linear alkyl benzene sulphonate; alkyl sulphates; alkyl ether sulphates and mixtures thereof. Preferably the alkyl ether sulphate is a C12-C14 n-alkyl ether sulphate with an average of 1 to 3EO (ethoxylate) units. Sodium lauryl ether sulphate is particularly preferred (SLES). Preferably the linear alkyl benzene sulphonate is a sodium C11 to C15 alkyl benzene sulphonates. Preferably the alkyl sulphates is a linear or branched sodium C12 to C18 alkyl sulphates. Sodium dodecyl sulphate is particularly preferred, (SDS, also known as primary alkyl sulphate).

The level of anionic surfactant in the detergent composition is preferably from (i) 5 to 50 wt % negatively charged surfactant, preferably the level of negatively charged surfactant is from 6 to 30 wt %, more preferably 8 to 20 wt %. Preferably two or more anionic surfactant are present, preferably linear alkyl benzene sulphonate together with an alkyl ether sulphate. Non-ionic surfactant may be present in the surfactant mix. Suitable nonionic detergent compounds which may be used include, in particular, the reaction products of compounds having an aliphatic hydrophobic group and a reactive hydrogen atom, for example, aliphatic alcohols, acids or amides, especially ethylene oxide either alone or with propylene oxide. Preferred nonionic detergent compounds are the condensation products of aliphatic Cs to Cie primary or secondary linear or branched alcohols with ethylene oxide. Preferably the alkyl ethoxylated non-ionic surfactant is a Cs to Cie primary alcohol with an average ethoxylation of 7EO to 9EO units.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

Materials and Methods

Chemical Reagents

The following analytical grade chemical compounds were purchased: pure 1,3 dodecanediol powder (custom manufactured by ZylexaPharma®, United Kingdom), chlorosulfonic acid 99% (by SigmaAldrich®, Ireland), 98% powder sodium borohydride (by SigmaAldrich®, Ireland), 99% HPLC grade Methanol (by Fisher Scientific®, Ireland). Tert-butanol (100%) (by Fluke®, Ireland), Diethyl Ether (99%) (by Fisher Scientific, Ireland), Magnesium Sulfate anhydrous powder (by SigmaAldrich®, Ireland), sodium chloride powder (by SigmaAldrich®, Ireland).

Biopolyester (polyhydroxylkanoate) Production

A strain of Pseudomonas chlororaphis 555, was cultivated in a stirred tank reactor using a fed-batch fermentation process. The inoculum (400 mL) was grown at 30° C. and 200 RPM in a shaking incubator for 20 hours in a minimal media (MSM), using hydrolysed rapeseed oil as carbon source. Hydrolysed plant oils were fed to bacterial cells in the bioreactor (Sartorius® B+ model), with a 5 litre working volume capacity. Buffering agents ammonia water (20% v/v) and sulphuric acid (15% v/v) were added to the bioreactor when required to maintain the pH at 7±0.05. The pH and dissolved oxygen (DO) were monitored during the fermentation by online probes. The air flow and the agitation rate (RPM) in the bioreactor were operated to maintain a dissolved oxygen above 20% of saturation in the growth media. The accumulated data were recorded into BioPAT® MFCS SCADA fermentation software (Sartorius AG, Germany).

Biopolyester Extraction (Downstream Process)

Microbial cells were harvested by centrifugation from the liquid culture media and freeze-dried using a Labconco® (Fisher Scientific) freeze-dryer. The total biomass was suspended in acetone in a ratio of 1:5 w/v for 24 hours. After the polymer dissolved in the solvent, the acetone fraction was filtered by vacuum filtration and most of the acetone was evaporated by rotary evaporation until approximately 20 mL of acetone containing polymer was left. This solution was added to 200 mL of −80° C. ethanol to precipitate the polymer. After the precipitation, the polymer was spread out in a stainless-steel tray to evaporate the residual solvents in a fume hood.

Methanolysis

The mcl-PHA polymer was methanolysed (coincident hydrolysis and methylation) by addition of methanol and sulfuric acid (85:15 ratio). The building blocks of the polymer were isolated as hydroxyfatty acid methyl esters (HFAME). The mixture of HFAME were analysed by gas chromatography-flame ionization detector (GC-FID using a HP-INNOWAX capillary column of 25 m×0.25 mm, with a 0.32 μm film thickness) as previously reported (Walsh et al 2015).

Chemical Reduction

The 3-hydroxyl moiety of HFAME was reduced at the carbonyl functional group by sodium borohydride (molar ratio NaBF₄:HFAME 2:1) as described by Soai et al 1984 and Dierker et al 2010 generating alkyl (1,3) diols. The chemical reaction was performed in tert-butanol with an excess of methanol as the hydrogen donor molecule.

Double Sulfation

1,3 alkyl diols (containing approx. 40% 1,3 decanediol, 30% 1,3 octanediol, 2% 1,3 hexanediol and 20% 1,3 dodecanediol) were sulphated by chlorosulfonic acid addition in diethyl ether. Sodium hydroxide was added afterwards in equimolar amount to neutralise the alkyldisulfates and generate sodium alkyl disulfates.

FTIR (Fourier Transformed Infrared Spectroscopy)

The sodium alkyl disulfates produced were characterised by FTIR. The infrared spectra were obtained with a Perkin Elmer Spectrum 100 FTIR Spectrometer, in the wavenumber range of 4000-550 cm-1, with a spatial resolution of 1 cm-1, at room temperature.

13C and 1H-NMR

Nuclear Magnetic Resonance (NMR) was undertaken to identify the synthesized alkyldisulfates. A Bruker Avance AM-400 Ultrashield™ with 4 nucleus (Varian Inc.® Inova™ model) spectrometer in the pulse-Fourier transform mode was employed at a frequency of 250 MHz using glass tubes with CDCl3 and methanol-D4 solution. A distorsionless enhancement by polarization transfer (DEPT) was adopted for 13C-NMR, to have an unequivocal attribution of primary, secondary or tertiary carbons. Two-dimensional analyses of the 13C and 1H NMR spectra were also performed: 2D homonuclear 1H-1H gradient Correlation spectroscopy (1H-1H COSY), Heteronuclear single-quantum correlation spectroscopy (130-1H HSQC) and Heteronuclear multi-bond correlation (1H-13C HMBC). These data were interpreted with MNova® MestreLab®; Chemical shifts (δ) are reported in ppm and coupling constants are given in Hz.

Drop Shape Analysis

The dynamic behaviour of a 20 μL deionised water drop after the addition of an equal volume of surfactant solution at critical micelle concentration was recorded with a video recording system and analysed by a dedicated plug-in (LB-ADSA) of ImageJ® software for image analysis. The sessile drop is positioned on a smooth and plan surface of a borosilicate glass slide without any microdefects. A contour recognition is initially carried out based on a grey-scale analysis of the image. In the second step, a geometrical model describing the drop shape is fitted to the contour. The contact angle is the angle between the calculated drop shape function and the sample surface.

Surface Tension Analysis and Critical Micelle Concentration (CMC)

Surface tension analysis was performed with an interfacial tensiometer Cenco DuNOUY® 70545 model. In this methodology, a ring-shaped steel tool is pulled up from a surfactant solution and the corresponding millinewton per meter (mN/m) of force applied to break the surface tension is indicated. Increasingly diluted surfactant solutions are measured until the reference value of deionised water is reached (72 mN/m). The measurements were performed at 25° C.

Critical micelle concentration was estimated by a conductivity assay. In this assay a pen type EC-963 model conductivity meter tester was submerged into a milliQ® grade deionised water solution to have a zero reading of pS/cm. Increasing amounts of different surfactant solutions was added to this milliQ® water solution and the conductivity was measured at 25° C. The increasing conductivity is proportional to the surfactant activity. The change of slope in the two linear interpolating curves is an indication of the critical micelle concentration (CMC) point for the specific surfactant (Al-Soufi et al 2012).

Foaming Stability

Equal volumes of three different anionic surfactants, at the same concentration (w/v): sodium alkyldisulfate, sodium dodecyldisulfate and sodium dodecylsulfate (SDS) were vortexed at constant stirring (1500 rpm) (with a VelpScientifica®, IR T4 model) for ten seconds and left to settle inside graduated test tubes to see the volume of the generated foam and the dynamic behaviour of the foam over time.

Results

Biopolymer and Hydroxyfatty Acid Methyl Ester (HFAME) Production

Fatty acids from hydrolysed plant origin were used as unique carbon source for the production of polyhydroxyalkanoate polymer by bacteria as reported by Walsh et al (2015). The PHA contained a mixture of (R)-3-hydroxyalkanoic acid monomers namely (R)-3-hydroxydodecanoic acid, (R)-3-hydroxydecanoic acid, (R)-3-hydroxyoctanoic acid, and (R)-3-hydroxyhexanoic acid in a ratio of 20:40:38:2. The polyhydroxyalkanoate polymer was used for the subsequent chemical reactions.

Methanolysis of polyhydroxyalkanoate produced hydroxyfatty acids methyl esters (HFAME) with 97% yield of reaction. The HFAME produced were analysed by GC-FID and confirmed the same monomer ratio (in mol %) as in the original polymer.

Analytical Grade 1,3 Dodecanediol Standard

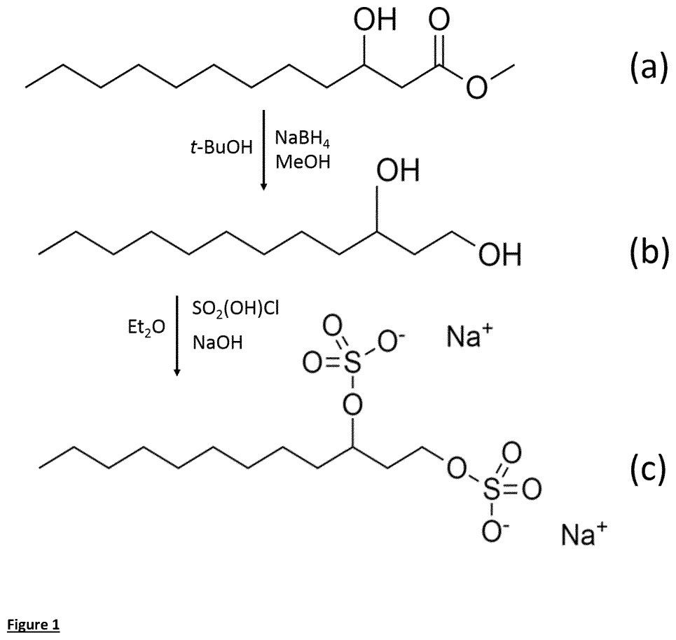

1,3 dodecanediol is the predicted product of the chemical reduction of (R)-3-hydroxydodecanoic acid. 1,3 dodecanediol was purchased by ZylexaPharma® and treated as a synthetic version of the chemically reduced HFAME. The 1H-NMR spectra of 1,3 dodecanediol was used as a reference (FIG. 1).

Double Sulfation of 1,3 Dodecanediol

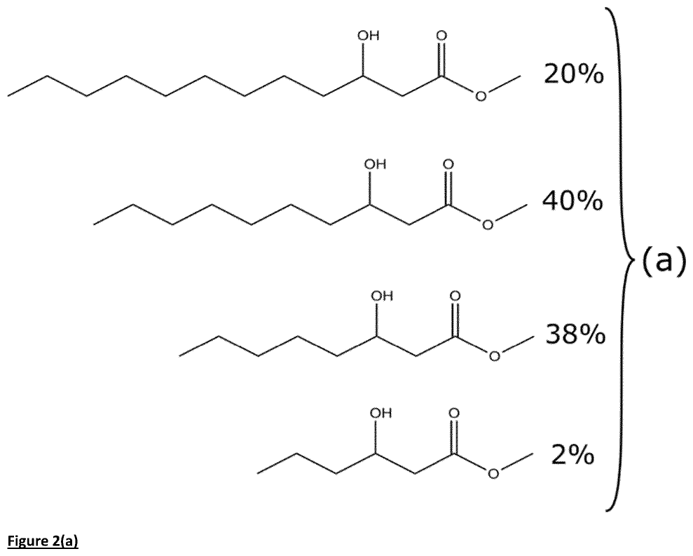

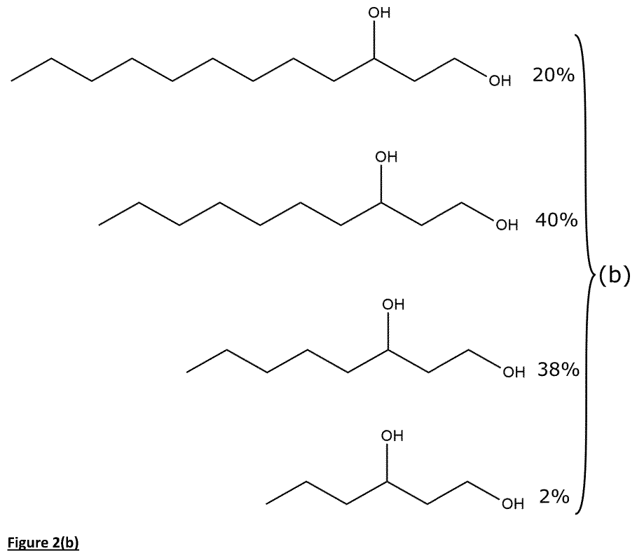

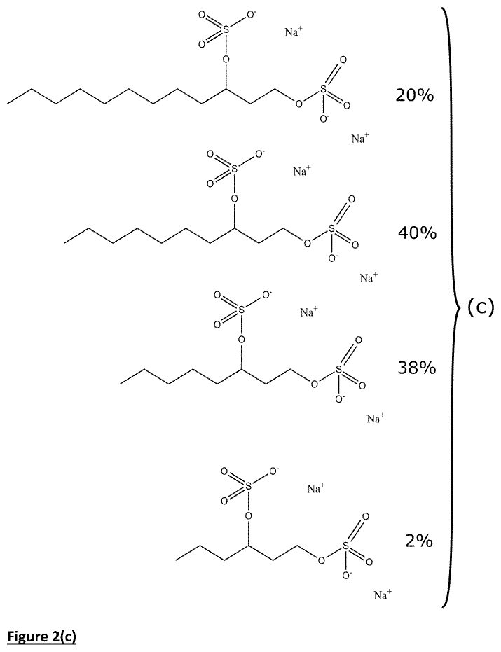

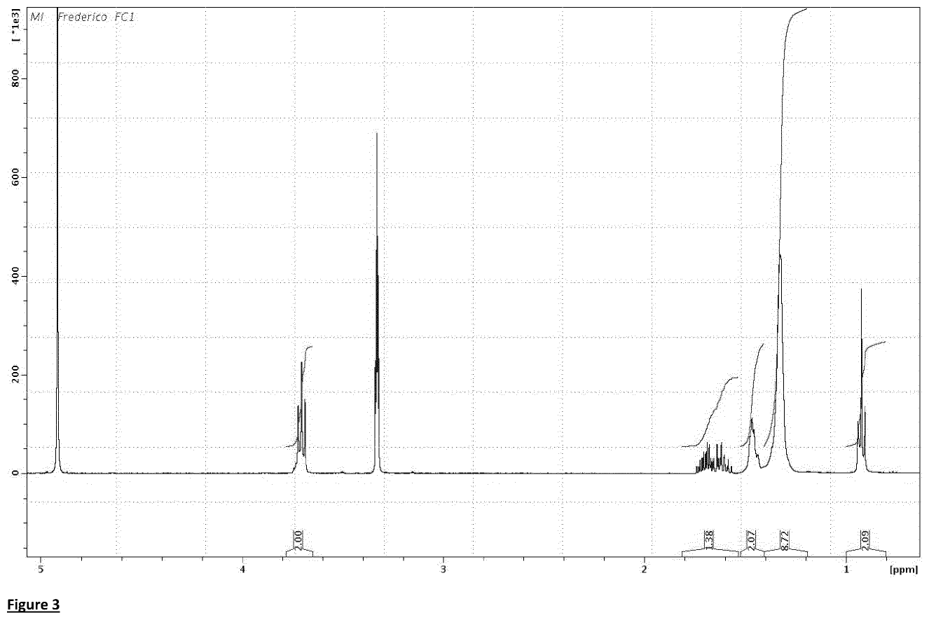

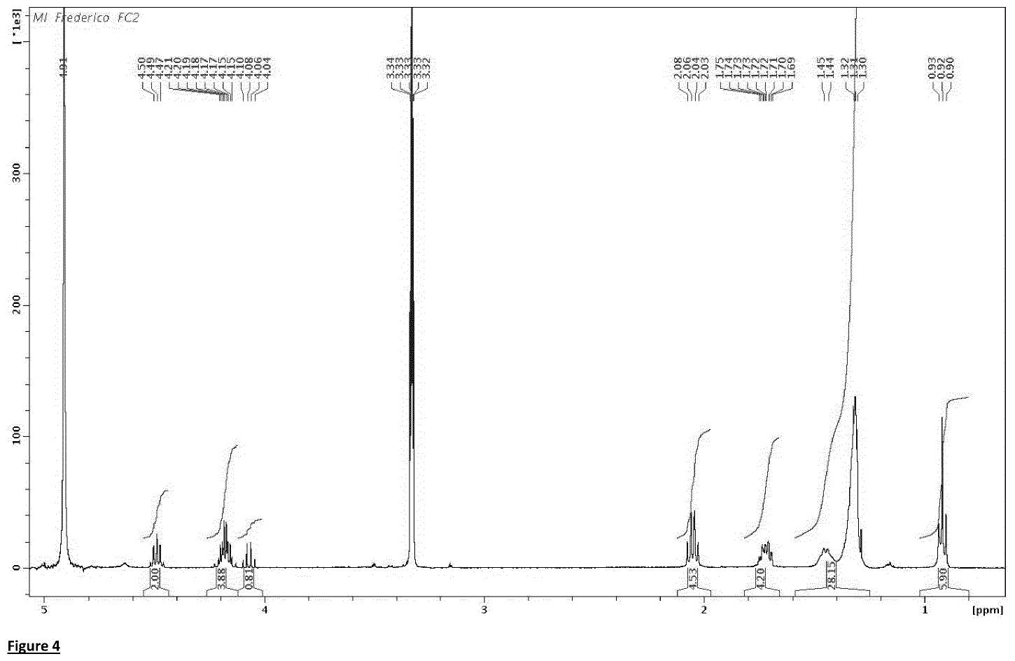

1,3 dodecanediol was dissolved in diethyl ether to allow the double sulfation of the two —OH residues by chlorosulfonic acid, similar to the method described by Dierker et al 2010. The absence of water allows the reaction to progress towards a complete disulfation of the 1,3 dodecanediol molecule. The chemical characterisation was performed using 13C, 1H NMR and FTIR. In the 1H NMR spectra, it can be seen that the peaks split at 4.1 and 4.4 ppm, respectively identify the hydrogens bound to two carbons involved in the C—O—S of the two sulfate groups in the dodecyl (1,3) disulfate molecule (FIG. 2) The 4.1 ppm peak is a triplet (t) and 4.4 ppm is a triplet of triplets (tt) coupling. The two-dimensional 1H COSY analysis confirms the interaction of the hydrogen bound to the first carbon and the hydrogens in the CH2 group of the second carbon; again the 4.4 ppm (tt) peak shows the coupling of this hydrogen (bound to the third carbon) with the hydrogens bound to the second carbon, located between the two sulfates groups. (FIG. 3) The 13C NMR also confirms the double sulfation of the molecule; this is particularly evident by the DEPT analysis of the 13C NMR, where the C in the methylene (CH2) group involved in the primary sulfate group bond (C—O—S bond) is found at 68 ppm (as predicted). Furthermore, the C in the CH group is located further downfield (77 ppm), because it is involved in the sulfate group resulting from the reaction with the secondary alcohol in the internal C—O—S. The HSQC confirms what we saw previously, the two protons shifted downfield at 4.1 and 4.4 ppm are unequivocally attributed to the carbon at 68 (CH2) and 77 (CH) ppm, respectively. (FIG. 4).

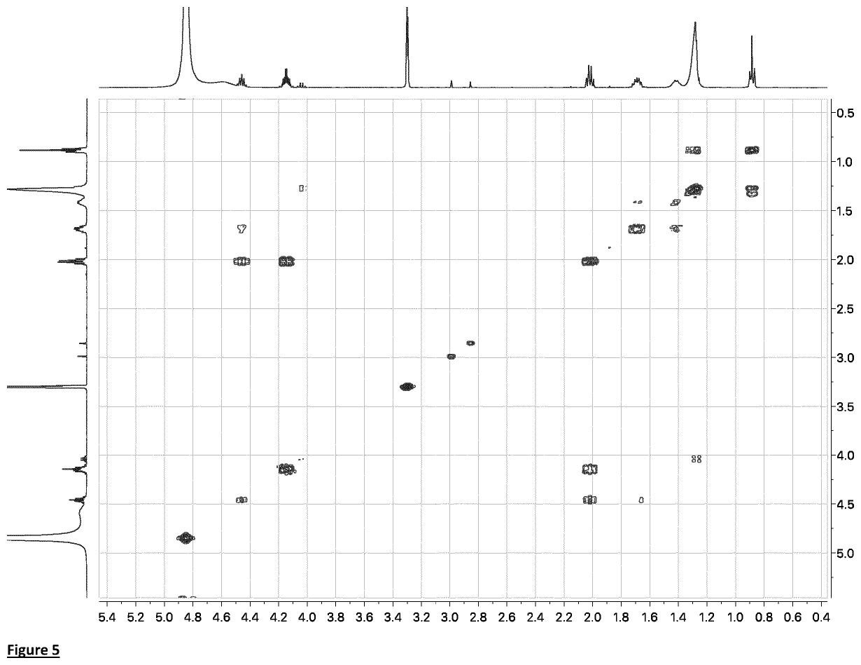

Analysing in detail the FTIR spectra (FIG. 5) we can see that many peaks confirm the methylene antisymmetric and symmetric vibrations at 2957 cm-1, 2851 cm-1, and 2919 cm-1 for alkyl CH stretching and 1465 cm-1 for alkyl CH deformation, respectively. From the FTIR spectra we can see that many peaks confirm the presence of the sulfate groups in the molecule; the absorption band at 824 cm-1 identifies the symmetrical vibration of C—O—S in the Co—O—SO3 group. Furthermore, the presence of another adsorption band at 848 cm-1, could also indicate the contribution of two different sulphate groups when bonded to two different oxygen in the ys C—O—S vibration. The absorption band at 1226 cm-1 is attributed to asymmetric (yas(E))S—O stretching mode. The same author also noticed the effect of the counterion in causing the shift of the absorption band to lower values compared to without the counterion. In particular the asymmetric (yas(A))S—O stretching mode and the symmetric (ys(A))S—O stretching mode both move to a lower wavenumber in presence of the counterion. In fact, the absorption bands at 1212 cm-1 and 1067 cm-1 can be caused by this feature (FIG. 5). Two very important absorption bands also prove the structure of the dodecyldisulfate molecule: The absence of any peak at 1700-1720 cm-1 specific for the carbonyl functional group (C═O bond) (already reduced in the upstream chemical reaction) and the presence of an absorption band at 1148 cm-1 that is usually attribute to C—O bond stretching. The neutralisation of the alkyl disulfates with equimolar NaOH is a critical step to prevent the reaction reversing. The aqueous solution of sodium alkyl disulfates is therefore stable and the compound does not revert to the diol and sulfuric acid when neutralised. This chemical synthesis protocol was adopted to convert the selected polyhydroxyalkanoate derived HFAME mixture into novel alkyldisulfate based bio-based surfactant.

Chemical Modification on HFAME Moiety to Produce 1,3 Alkyldiols

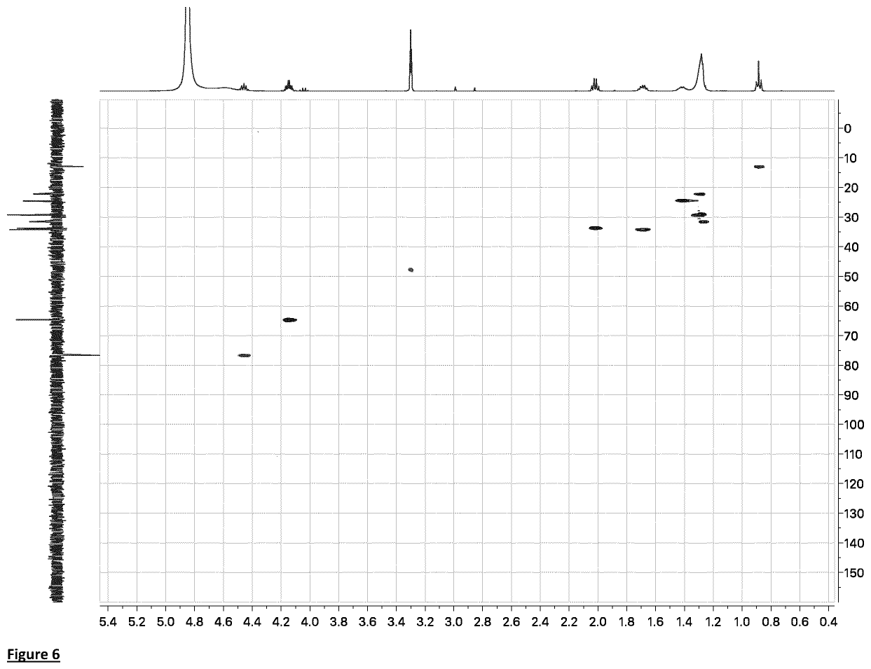

HFAME, arising from PHA methanolysis, were reduced by sodium borohydride (NaBH4) as described by Soai et al., (1984) in tert-butanol with an excess of methanol as the coordinating compound for the proton donation. 1,3 alkyldiols were obtained with a 70% yield and the structure was confirmed by comparing it with 1,3 dodecanediol 1H-NMR and by the FTIR spectra. (FIG. 6).

Double Sulfation of Diol Moiety

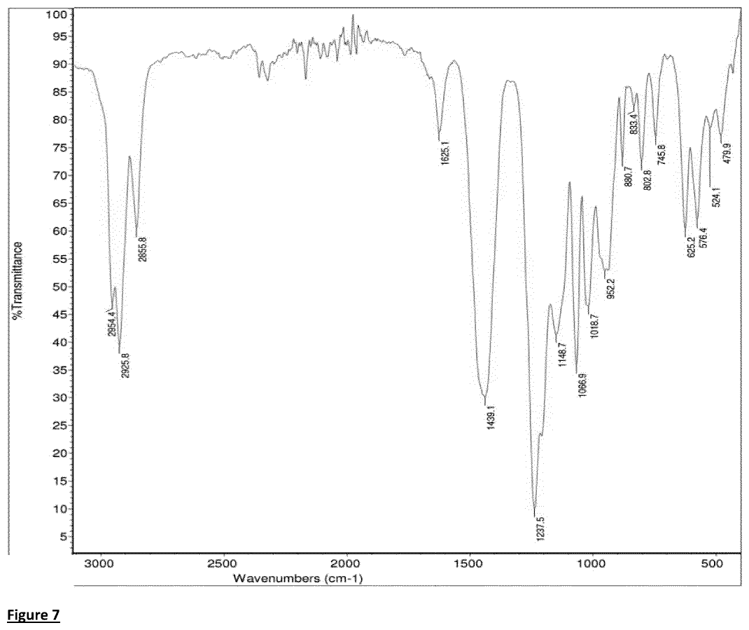

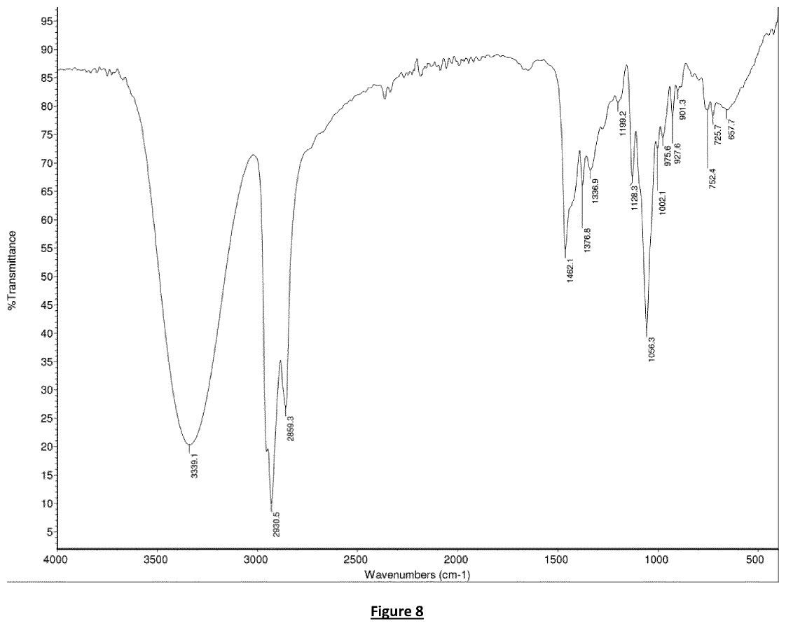

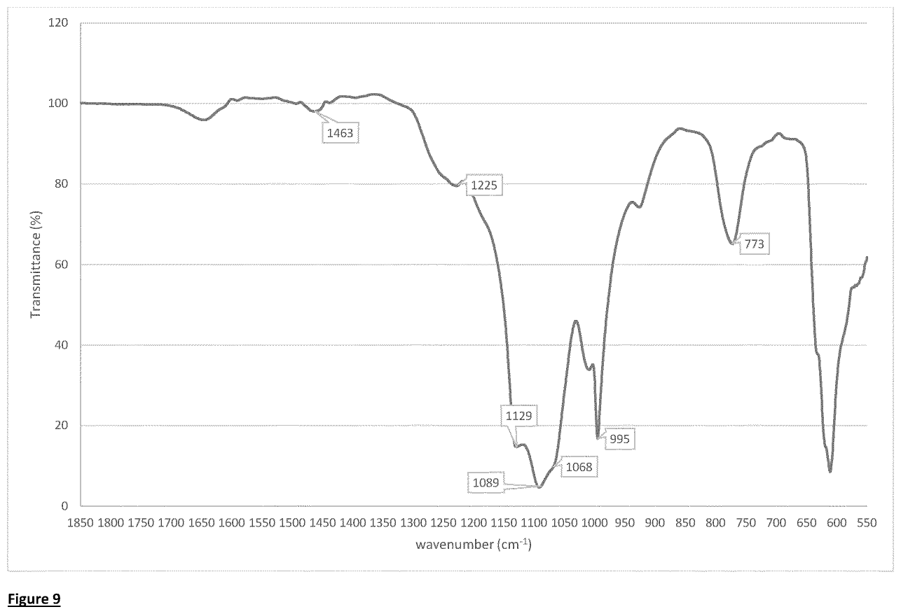

The 1,3 alkyldiols were dissolved in diethyl ether to allow the double sulfation of the two —OH residues by chlorosulfonic acid. The procedure was performed as done by Dierker et al 2010. The absence of water is critical to allow the reaction to progress towards complete disulfation of the 1,3 alkyldiol moiety. Therefore, an excess of calcium chloride was used to make sure no water affected the reaction. The reaction mixture containing the alkyldiol products was neutralised by equimolar sodium hydroxide to produce sodium alkyl disulfates. FTIR spectra (FIG. 7) shows similarities with dodecyl (1,3) disulfate spectra with peaks that confirm the methylene antisymmetric and symmetric vibrations at 2957 cm-1, 2851 cm-1, and 2919 cm-1 for alkyl CH stretching and 1465 cm-1 for alkyl CH deformation, respectively. The absorption band at 1089 cm-1 with a shoulder at 1068 cm-1 could be attributed to the symmetric (ys(A))S—O stretching mode. At the same time, the asymmetric (yas(A))S—O stretching mode is also present with an absorption band at 1225 cm-1. The presence of an absorption band at 773 cm-1 seems too low to be the C—O—S vibration (usually found in the 800-850 cm-1 region) but, according to Prosser and co-workers (2002) a sharp absorption band we observe at 1000 cm-1 can also be attributed to the C—O—S vibrations. The complete absence of an absorption band in the region 1720-1730 cm-1 confirms unequivocally the reduction of the carbonyl functional group. 13C and 1H-NMR were also performed on this alkyldisulfate moiety. According to the 1H-COSY, the usual peak at 4.1 ppm is appearing weakly in this case but the peak at 4.7 ppm (as a multiplet peak close to the bigger peak of the hydroxyl of deuterated methanol is also coupling with the peak at 2.3 and 2.7 ppm as it shown in the cross-peaks (FIG. 8). The 13C NMR is less resolved than the cleaner (synthetic origin) dodecyldisulfate, but still shows the usual peak at 77 ppm belonging to the C in the CH group involved in the C—O—S bond of the original secondary alcohol. (FIG. 9). The lower resolution is due to the fact that the PHA derived alkyldisulphates contain a mixture of alkyl chain lengths.

Surface Tension

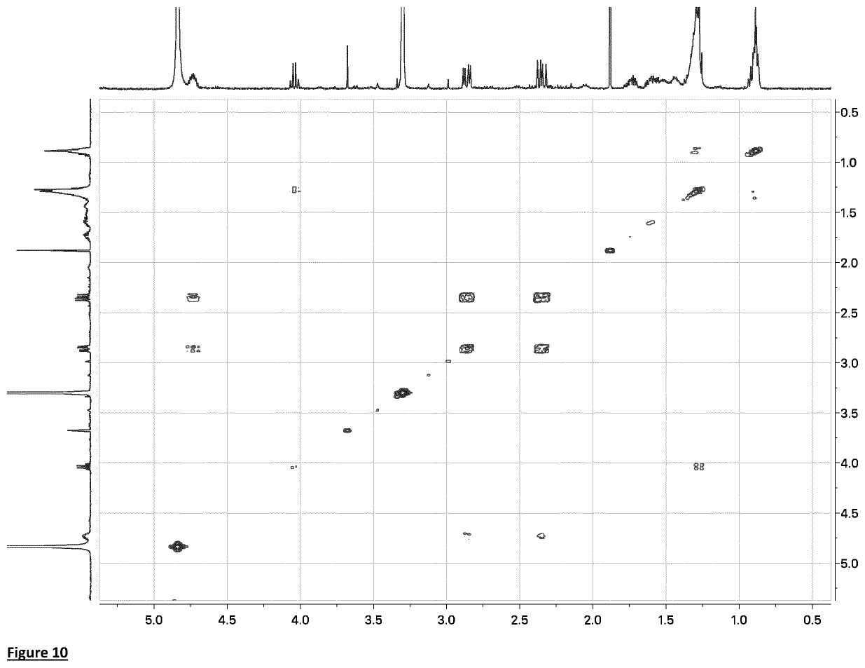

A solution of a commercial purchased dodecyl (1,3) disulfate was progressively diluted by doubling the amount of deionised water until it reached the literature reference value of surface tension for pure deionised water (72 mN/m-1) (FIG. 10). At parity of concentration (w/v) the surface tension value for dodecyl (1,3) disulfate is 4-fold better than sodium dodecyl sulfate (SDS). The alkyl disulfate generated from the sulphation of PHA derived 3-hydroxyalkanoic acids methyl esters perform 16-times better than SDS at the same concentration (w/v). It is possible that there is a synergic effect of the different alkyl chains to increase the surfactant properties of the mixture with respect to dodecyl (1,3) disulfate alone.

Critical Micelle Concentration (CMC)

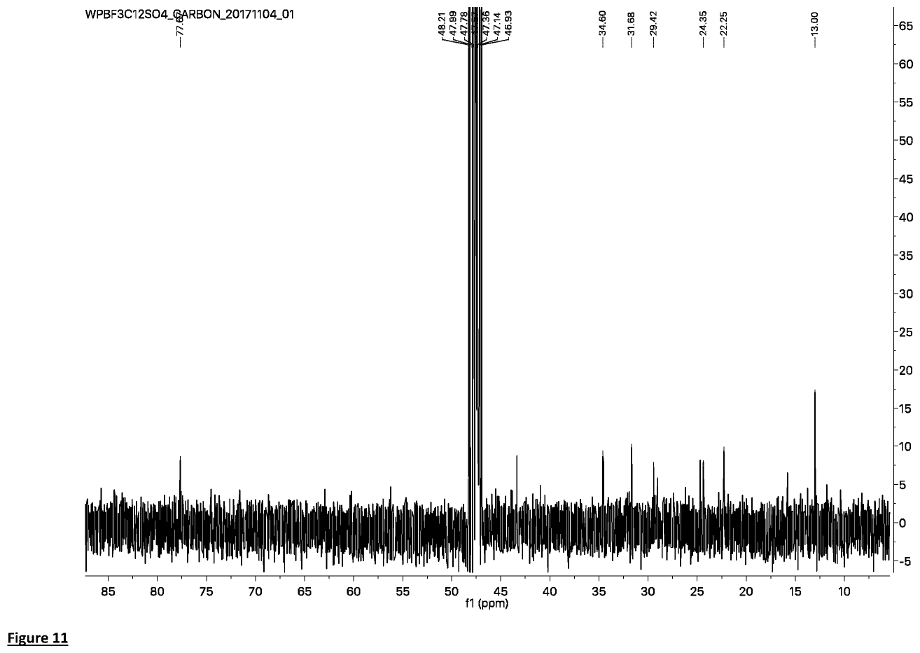

The critical micelle concentration is derived from the surface tension curve of the compound and is at a point where an increase in the concentration of the surfactant does not increase the ability to form micelles. When this concentration was known it was then possible to conduct another set of tests to confirm the ability of the alkyldisulfate mixture, derived from the PHA monomers to act as surfactants. The trend in conductivity values of increasing concentration of surfactants is shown in FIG. 11. It can be seen that dodecyl (1,3) disulfate outperform SDS by 3.5-fold at the specific CMC concentration (change of slope point) while the PHA derived alkyldisulfate moiety is 6.1-fold better at the same specific CMC, compared to SDS. All the respective interpolating curves exhibit an R2 value close to 1, that is an indication of the correct interpolation of the curves. The more efficient performance of the PHA derived alkyldisulfate could be attributed to the longer and shorter alkyl chains which would increase the hydrophilicity-hydrophobicity ratio and thus allow a better performance of the surfactant. The presence of multiple anionic polar heads is the core nature of another type of surfactants: the gemini surfactants, in these there is a specific combined feature of multiple polar heads together with a long enough aliphatic chain (C>12) to increase the surfactant properties of the compound. A similar phenomenon, can be hypothesized in the bio-based surfactants of the current study.

Drop Analysis (Wettability)

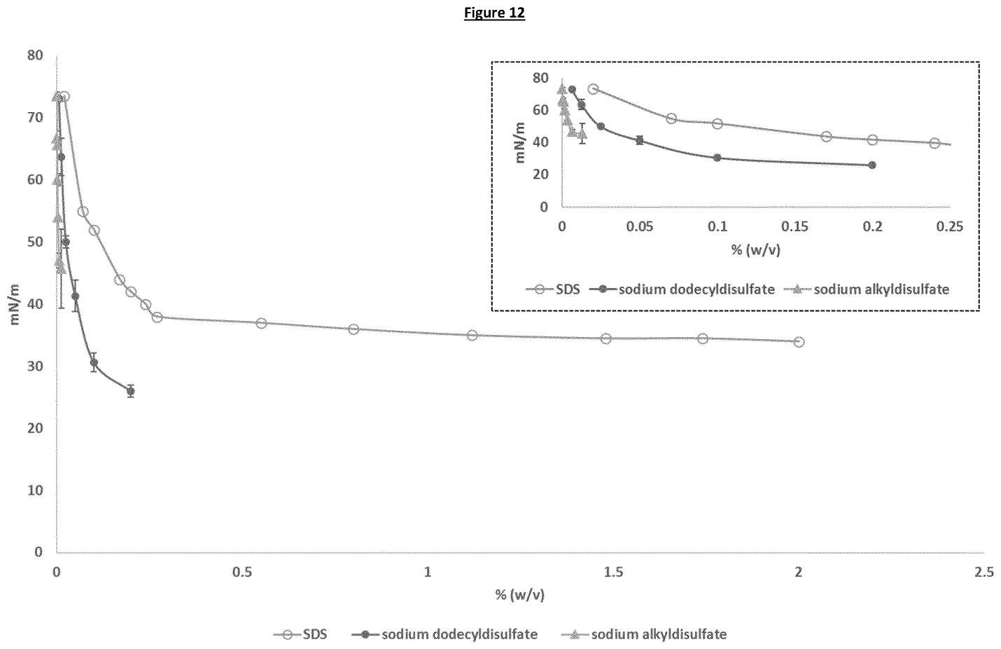

When a dodecyl (1,3) disulfate solution at its CMC is added to an equivalent volume of deionised water (a drop of 20 μL), the spreading of the solution on a flat surface allows for the calculation of the dynamic contact angle evolution over time (Supplementary video 1). The wettability (speed at which the contact angle of a deionised single drop of water is broken over time) of the dodecyl (1,3) disulfate solution is higher that the SDS solution (FIG. 12). The evolution of the contact angle of the PHA derived dialkylsulphate over time is 18% fold slower than its synthetic version (dodecyl (1,3) disulfate) but still 9% faster than SDS (Supplementary video 2). The control is an equivalent volume of deionised water which is added to the same drop of deionised water where the contact angle evolution is almost a flat line (FIG. 12).

Foaming Stability

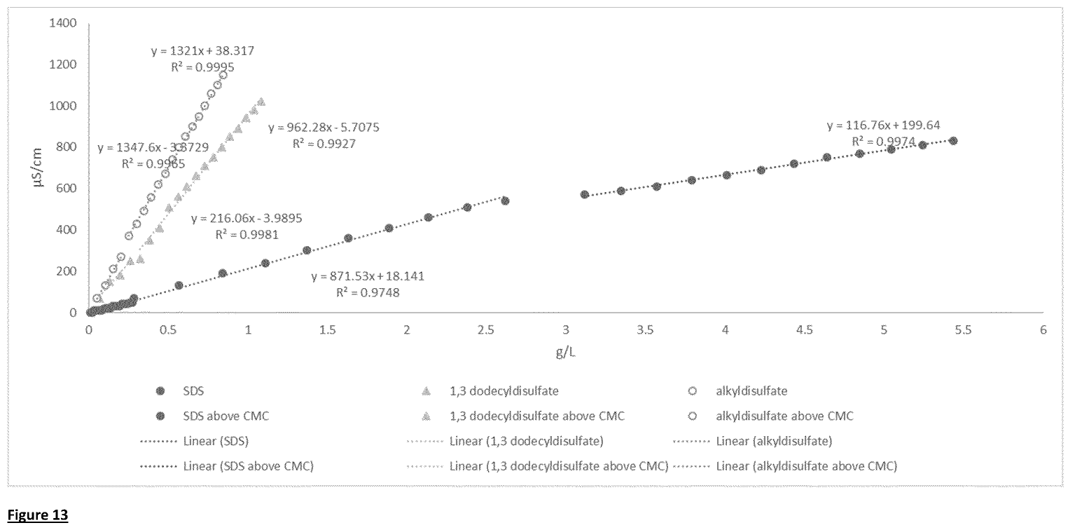

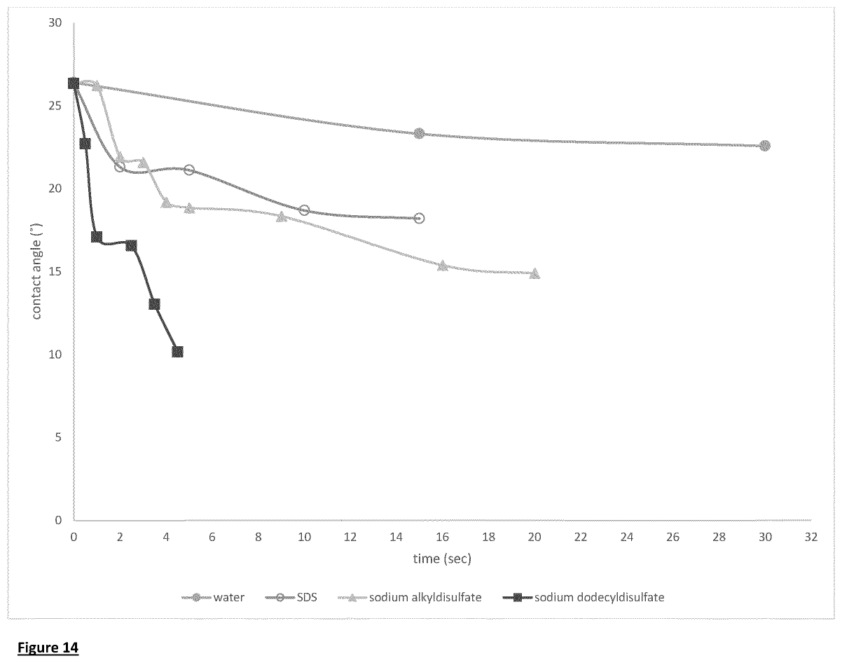

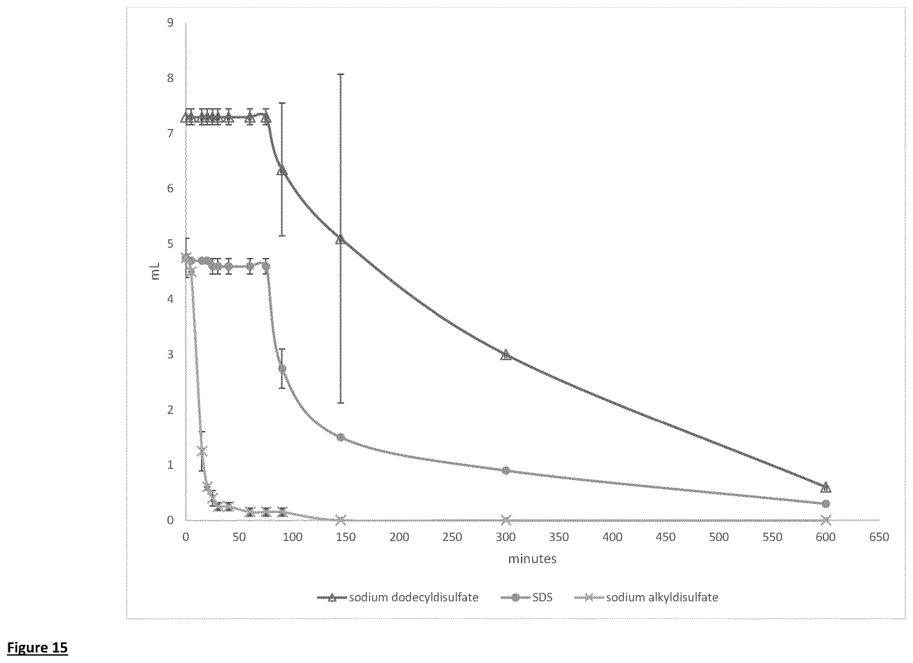

Another known property of surfactants is the ability to form and maintain a stable foam after a period of constant stirring. To evaluate this effect, we performed a 10-seconds stirring at 1500 rpm and evaluated the decrease of the foam volume over time (FIG. 13). It is evident that the dodecyldisulfate surfactant causes a more sustained volume of foam after shaking. The foaming volume is 1.5 fold higher than the SDS and the foam is more stable over time decreasing 2 fold in volume and 3 times slower than SDS at their respective CMC values. The PHA derived alkyldisulfates, even if showing better surfactant properties, have a higher foaming ability compared to SDS but lower than dodecyldisulphate at the CMC concentrations. The Marangoni counterflow that stabilises the bubble stability in the lamella region, due to the gradient movement of surfactants molecules might be easily achievable with a homogenous composition of dodecyldisulfates. However the presence of alkydisulfates of different chain length might introduce a weaker Marangoni effect and the predominating plateau border flow causes a faster coalescence of the lamella and the collapse of the bubbles.

The highest standard error occurred at 145 minutes for the sodium dodecylsulfate surfactant. This can be explained by the fact that at this particular time only a few remaining bubbles sustain the foam structure that was generated at TO.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

### REFERENCES

M. Walsh, K. O'Connor, R. Babu, T. Woods, S. T. Kenny. Plant Oils and Products of Their Hydrolysis as Substrates for Polyhydroxyalkanoate Synthesis Chem. Biochem. Eng. Q. 29 (2015) 123-133
K. Soai, H. Oyamada, M. Takase. The preparation of N-protected amino alcohols and N-protected peptide alcohol by reduction of the corresponding esters with sodium borohydride. An improved procedure involving a slow addition of a small amount of methanol. Bull. Chem. Soc. Japan 57 (1984) 2327-2328
M. Dierker, S Hans Surfactants from oleic, erucic and petroselinic acid: Synthesis and properties Eur. J. lipid Sci. Technol. 112 (2010) 122-136
W. Al-Soufi, L Piñeiro, M. Novo 2012 A Model for Monomer and Micellar Concentrations in Surfactant Solutions. Application to Conductivity, NMR, Diffusion and Surface Tension data. J Colloid Interf Sci 370: 102-110
S. Y. Lee, H. H. Wong, J. I., Choi, S. H. Lee, C. S. Han 2000 Production of medium-chain-length polyhydroxyalkanoates by high-cell-density cultivation of Pseudomonas putida under phosphorus limitation. Biotechnol Bioeng 20: 466-470
Madison et al (Microbiology and Molecular Biology Reviews, March 1999, P21-53)

1. A process for producing a bio-based surfactant comprising alkyl disulphate salts comprising the steps of:
methanolysis of medium chain length polyhydroxyalkanoic acid (mcl-PHA) to provide hydroxy fatty acid methyl ester monomers (HFAME's);
reduction of the HFAME's to provide 1,3 alkyl diols;
sulphation of the 1,3 alkyl diols to provide 1,3 alkyl disulphates; and
neutralisation of the alkyl disulphates to provide a bio-based surfactant comprising 1,3 alkyl disulphate salts.

2. A process according to claim 1, including an initial step of microbial fermentation of fatty acids to produce the mcl-PHA.

3. A process according to claim 2, in which the fatty acids are of hydrolysed plant oil origin.

4. A process according to any preceding claim, in which the methanolysis step employs methanol and sulphuric acid.

5. A process according to any preceding claim, in which the reduction step employs a borohydride salt, and/or in which the neutralisation employs a sodium hydroxide and in which the alkyl disulphate salt is sodium alkyl disulphate.

6. A process according to any preceding claim, in which the mcl-PHA comprises C-12 hydroxyalkanoic acid in at least 15 mol % in the polymer.

7. A process according to any preceding claim, in which the mcl-PHA comprises C-6, C-8, C-10 and C-12 hydroxyalkanoic acids.

8. A process according to any preceding claim, in which the mcl-PHA consist essentially of C-6, C-8, C-10 and C-12 hydroxyalkanoic acids.

9. A process according to claim 8, in which a mol % ratio of C-6, C-8, C-10 and C-12 hydroxyalkanoic acids in the mcl-PHA polymer is about 1-10:10-60:10-60:15-79.

10. A bio-based surfactant comprising a mixture of medium chain length 1,3 alkyl disulphate salts and prepared by a process comprising methanolysis (depolymerisaion) of mcl-PHA to produce hydroxy fatty acid methyl esters (HFAME's), which are reduced and sulphated to provide a mixture of alkyl disulphates, and then neutralised to provide the bio-based surfactant comprising a mixture of medium chain length 1,3 alkyl disulphate salts

11. A bio-based surfactant according to claim 10, comprising C6, C8, C10 and C12 1,3 alkyl disulphate salts.

12. A bio-based surfactant according claim 11, consisting essentially of C6, C8, C10 and C12 1,3 alkyl disulphate salts.

13. A bio-based surfactant according to any of claims 10 to 12, in which C12 alkyl disulphate salt comprises at least 15 mol % of the mixture.

14. A bio-based surfactant according to any of claims 10 to 13, in which a mol % ratio of C6, C8, C10 and C12 alkyl disulphate salts in the mixture is about 1-10:10-60:10-60:15-79.

15. A bio-based surfactant according to claim 14, in which a mol % ratio of C6, C8, C10 and C12 alkyl disulphate salts in the mixture is about 1-5:30-50:30-50:15-39.

16. A composition comprising a bio-based surfactant according to any of claims 10 to 15.

17. A composition according to claim 16, which is a detergent composition.

18. A composition according to any of claim 16 or 17, in which the composition additionally comprises one or more of a non-ionic surfactant, additional anionic surfactant, and a co-surfactant.

19. A composition according to claim 18, in which the co-surfactant is selected from an amphoteric surfactant, a zwitterionic surfactant, a cationic surfactant, or a mixture thereof.

20. A composition according to any of claims 16 to 19, in which the composition is selected from the group consisting of: a personal care product; a fabric washing product; a dishwashing product; a household care product; a liquid, solid or semi-solid soap; a fabric washing product; a dishwashing product; a shampoo; a shower or body gel; a household cleaning detergent; and a toothpaste.
